(19) United States
(12) Patent Application Publication
(10) Pub. No.: US 2003/0078374 A1
(43) Pub. Date: Apr. 24, 2003

(54) Complementary peptide ligands generated from the human genome

(76) Inventors: Gareth W. Roberts, Cambridge (GB); Jonathan R. Heal, Highbury (GB)

Correspondence Address: FINNEGAN, HENDERSON, FARABOW, GARRETT &, DUNNER LLP, 1300 I STREET, NW, WASHINGTON, DC, 20006, US

(21) Appl. No.: 09/572,404

(22) Filed: May 17, 2000

(30) Foreign Application Priority Data
Dec. 13, 1999 (GB) 9929464.7

(51) Int. Cl. G06F 19/00; G01N 33/48; G01N 33/50; C07H 21/04

(52) U.S. Cl. 530/350; 536/23.2; 702/19

(57) ABSTRACT

In the current invention the application of our novel informatics approach to the databases containing nucleotide and peptide sequences from the human genome generates the sequence of many peptides which form the basis of an innovative and novel approach to developing new therapeutic agents.

This invention claims the use of specific complementary peptides to the proteins encoded in the human genome as reagents and drugs for drug discovery programmes.

Specific protein interactions are critical events in most biological processes in health and disease. A clear idea of the way proteins interact, their three dimensional structure and the types of molecules which might block or enhance interaction are critical aspects of the science of drug discovery in the pharmaceutical industry.

Current predictions estimate that the human genome will be sequenced by 2002 if not sooner. This has accelerated the requirement for informatics tools for mining of the genomic sequence data. A process for the searching and analysis of protein and nucleotide sequence databases has been identified. Significant utility can be acheived within the pharmaceutical i industry by searching and analysing protein and nucleotide sequence databases to identify complementary peptides that interact with their relevant target proteins.

These novel peptides can be used as lead ligands to facilitate drug design and development. This invention describes the application of this process to the databases containing nucleotide and protein sequence data from the human genome.

This invention claims the use of specific complementary peptides to the proteins encoded in the human genome as reagents and drugs for drug discovery programmes.

### BACKGROUND

Specific protein interactions are critical events in most biological processes and a clear idea of the way proteins interact, their three dimensional structure and the types of molecules which might block or enhance interaction are critical aspects of the science of drug discovery in the pharmaceutical industry.

Proteins are made up of strings of amino acids and each amino acid in a string is coded for by a triplet of nucleotides present in DNA sequences (Stryer 1997). The linear sequence of DNA code is read and translated by a cell's synthetic machinery to produce a linear sequence of amino acids that then fold to form a complex three-dimensional protein.

In general it is held that the primary structure of a protein determines its tertiary structure. A large volume of work supports this view and many sources of software are available to the scientists in order to produce models of protein structures (Sansom 1998). In addition, a considerable effort is underway in order to build on this principle and generate a definitive database demonstrating the relationships between primary and tertiary protein structures. This endeavour is likened to the human genome project and is estimated to have a similar cost (Gaasterland 1998).

The binding of large proteinaceous signaling molecules (such as hormones) to cellular receptors regulates a substantial portion of the control of cellular processes and functions. These protein-protein interactions are distinct from the interaction of substrates to enzymes or small molecule ligands to seven-transmembrane receptors. Protein-protein interactions occur over relatively large surface areas, as opposed to the interactions of small molecule ligands with serpentine receptors, or enzymes with their substrates, which usually occur in focused “pockets” or “clefts”. Thus, protein-protein targets are non-traditional and the pharmaceutical community has had very limited success in developing drugs that bind to them using currently available approaches to lead discovery. High throughput screening technologies in which large (combinatorial) libraries of synthetic compounds are screened against a target protein(s) have failed to produce a significant number of lead compounds.

Many major diseases result from the inactivity or hyperactivity of large protein signaling molecules. For example, diabetes mellitus results from the absence or ineffectiveness of insulin, and dwarfism from the lack of growth hormone. Thus, simple replacement therapy with recombinant forms of insulin or growth hormone heralded the beginnings of the biotechnology industry. However, nearly all drugs that target protein-protein interactions or that mimic large protein signaling molecules are also large proteins. Protein drugs are expensive to manufacture, difficult to formulate, and must be given by injection or topical administration.

It is generally believed that because the binding interfaces between proteins are very large, traditional approaches to drug screening or design have not been successful. In fact, for most protein-protein interactions, only small subsets of the overall intermolecular surfaces are important in defining binding affinity.

“One strongly suspects that the many crevices, canyons, depressions and gaps, that punctuate any protein surface are places that interact with numerous micro- and macro-molecular ligands inside the cell or in the extra-cellular spaces, the identity of which is not known” (Goldstein 1998).

Despite these complexities, recent evidence suggests that protein-protein interfaces are tractable targets for drug design when coupled with suitable functional analysis and more robust molecular diversity methods. For example, the interface between hGH and its receptor buries ˜1300 Sq. Angstroms of surface area and involves 30 contact side chains across the interface. However, alanine-scanning mutagenesis shows that only eight side-chains at the center of the interface (covering an area of about 350 Sq. Angstroms) are crucial for affinity. Such “hot spots” have been found in numerous other protein-protein complexes by alanine-scanning, and their existence is likely to be a general phenomenon.

The problem is therefore to define the small subset of regions that define the binding or functionality of the protein.

The important commercial reason for this is that a more efficient way of doing this would greatly accelerate the process of drug development.

These complexities are not insoluble problems and newer theoretical methods should not be ignored in the drug design process. Nonetheless, in the near future there are no good algorithms that allow one to predict protein binding affinities quickly, reliably, and with high precision (Sunesis website www.sunesis.com Sep. 17, 1999).

A process for the analysis of whole genome databases has been developed. Significant utility can be achieved within the pharmaceutical industry by searching and analyzing protein and nucleotide sequence databases to identify complementary peptides which interact with their relevant target proteins.

These novel peptides can be used as lead ligands to facilitate drug design and development. This invention describes the application of this process to databases containing nucleotide and protein sequence data from the human genome.

The process has been described in patent application number GB 9927485.4, filed Nov. 19, 1999 for use in analysing, and manipulating the sequence data (both DNA and protein) found in large databases and its utility in conducting systematic searches to identify the sequences which code for the key intermolecular surfaces or “hot spots” on specific protein targets.

This technology will have significant applications in the application of informatics to sequence databases in order to identify lead molecules for numerous important pharmaceutical targets.

### THE INVENTION

In the current invention the application of our novel informatics approach to the databases containing nucleotide and peptide sequences from the human genome generates the sequence of many peptides which form the basis of an innovative and novel approach to developing new therapeutic agents.

This invention claims the use of specific complementary peptides to the proteins encoded in the human genome as reagents and drugs for drug discovery programmes.

### APPLICATION OF THE DATA MINING PROCESS TO THE ANALYSIS OF THE HUMAN GENOME

One of the key aims of the Human Genome Project is to identify all of the 80,000 to 140,000 genes in human DNA and to determine the complete sequence of the genome (3 billion bases). The first working draft of the human genome sequence (90% coverage) is likely to be completed by 2000 with the finished sequence being completed by 2002. The public availability of this sequence has provided a resource that can now be mined using novel informatics technologies.

Most human genes are expressed as multiple distinct proteins. It has been estimated that the number of actual proteins generated by the human genome is at least ten times greater. The data mining process described, patent application number GB 9927485.4 greatly accelerates the pace of identification and optimization of small peptides that bind to protein-protein targets. This provides a means of reducing the complexity of the human genetic information by identifying those regions of proteins that are likely to be important targets for drug development. In addition, the computational methods identify proteins that are functionally linked through different pathways or structural complexes.

We have applied our computational approach with its novel algorithms for generating complementary peptides, patent application number GB 9927485.4, to the human genome. Human nucleotide and protein sequence data is publicly available in a number of large databases (see EXAMPLE 1), and these are continually updated as more sequence becomes available. The identification of novel complementary peptides will allow new lead ligands to enhance drug design and discovery.

The biological relevance of this approach is described (EXAMPLE 2) and the utility of peptides as tools for functional genomics studies is outlined in EXAMPLE 3.

A catalogue of complementary inter-molecular peptides frame size 10 (average 3 per gene) was generated for each gene within the human genome (see EXAMPLE 4).

Sets of shorter ‘daughter’ sequences of frame size 5, 6, 7, 8 or 9 can also be derived from these sequences (EXAMPLE 5).

A further set of intra-molecular complementary peptide sequences was also generated for each gene within the human genome (see EXAMPLE 6).

Sets of shorter ‘daughter’ sequences of frame size 5, 6, 7, 8 or 9 can also be derived from these sequences (EXAMPLE 7).

Each complementary peptide sequence has a unique identifying number in the catalog and peptides are categorised as either intra-molecular or inter-molecular peptides within the human genome as shown in the table below (and in EXAMPLES 4 and 6):

| Genome | Inter-molecular peptides | Intra-molecular peptides |
|---|---|---|
| Human | 1-3622 | 3624-4203 |

Utilizing our novel approach we were able to discover the sequences of complementary peptides that have the potential to interact with and alter the functionality of the relevant protein coded for by its gene. Furthermore the second analysis provides information as to the regions on other proteins which might interact with the first protein (its ‘molecular partners’ in physiological functions).

The peptide sequences described in this patent can be readily made into peptides by a multitude of methods. The peptides made from the sequences described in this patent will have considerable utility as tools for functional genomics studies, reagents for the configuration of high-throughput screens, a starting point for medicinal chemistry manipulation, peptide mimetics, and therapeutic agents in their own right.

The process of patent application number GB9927485.4 will now be described below. The examples of this present application are the result of applying that process to a selected human database: it will readily be appreciated that use of the process on other databases will yield peptide sequences and catalogues of intra- and inter-molecular complementary peptides specific to the other human databases (e.g. the databases in EXAMPLE 1).

The current problems associated with design of complementary peptides are:

A lack of understanding of the forces of recognition between complementary peptides

An absence of software tools to facilitate searching and selecting complementary peptide pairs from within a protein database

A lack of understanding of statistical relevance/distribution of naturally encoded complementary peptides and how this corresponds to functional relevance.

Based on these shortfalls, our process provides the following technological advances in this field:

A mini library approach to define forces of recognition between human Interleukin (IL) 1β and its complementary peptides.

A high throughput computer system to analyse an entire database for intra/inter-molecular complementary regions.

Studies into preferred complementary peptide pairings between IL-1β and its complementary ligand reveal the importance of both the genetic code and complementary hydropathy for recognition. Specifically, for our example, the genetic code for a region of protein codes for the complementary peptide with the highest affinity. An important observation is that this complementary peptide maps spatially and by residue hydropathic character to the interacting portion of the IL-1R receptor, as elucidated by the X-ray crystal structure Brookhaven reference pdb2itb.ent.

Using these novel observations as guiding principles for analysis, we have developed a computational analysis system to evaluate the statistical and functional relevance of intra/inter-molecular complementary sequences.

This process provides significant benefits for those interested in:

The analysis and acquisition of peptide sequences to be used in the understanding of protein-protein interactions.

The development of peptides or small molecules which could be used to manipulate these interactions.

The advantages of this process to previous work in this field include:

Using a valid statistical model. Previously, complementary mappings within protein structures has been statistically validated by assuming that the occurrence of individual amino acids is equally weighted at 1/20 (Baranyi, 1995). Our statistical model takes into account the natural occurrence of amino acids and thus generates probabilities dependent on sequence rather than content per se.

Facilitation of batch searching of an entire database. Previously, investigations into the significance of naturally encoded complementary related sequences have been limited to small sample sizes with non-automated methods. The invention allows for analysis of an entire database at a time, overcoming the sampling problem, and providing for the first time an overview or ‘map’ of complementary peptide sequences within known protein sequences.

The ability to map complementary sequences as a function of frame size and percentage antisense amino acid content. Previously, no consideration has been given to the significance of the frame length of complementary sequences. Our process produces a statistical map as a function of frame size and percentage complementary residue content such that the statistical importance of how nature selects these frames may be evaluated.

### BRIEF DESCRIPTION OF DRAWINGS

The process is described with reference to accompanying drawings. In the drawings, like reference numbers indicate identical or functionally similar elements.

FIG. (1) shows a block diagram illustrating one embodiment of a method of the present invention

FIG. (2) shows a block diagram illustrating one embodiment for carrying out Step 4 in FIG. (1)

FIG. (3) shows a block diagram illustrating one embodiment for carrying out Step 5 in FIG. (1)

FIG. (4) shows a block diagram illustrating one embodiment for carrying out Step 8 in FIGS. (2) and (3)

FIG. (5) shows a block diagram illustrating one embodiment for carrying out Step 8 in FIGS. (2) and (3)

FIG. (6) shows a block diagram illustrating one embodiment for carrying out Step 6 in FIG. (1)

### A DESCRIPTION OF THE ANALYTICAL PROCESS

The software, ALS (antisense ligand searcher), performs the following tasks:

Given the input of two amino acid sequences, calculates the position, number and probability of the existence of intra- (within a protein) and inter- (between proteins) molecular antisense regions. ‘Antisense’ refers to relationships between amino acids specified in EXAMPLES 8 and 9 (both 5′->3′ derived and 3′->5′ derived coding schemes).

Allows sequences to be inputted manually through a suitable user interface (UI) and also through a connection to a database such that automated, or batch, processing can be facilitated.

Provides a suitable database to store results and an appropriate interface to allow manipulation of this data.

Allows generation of random sequences to function as experimental controls.

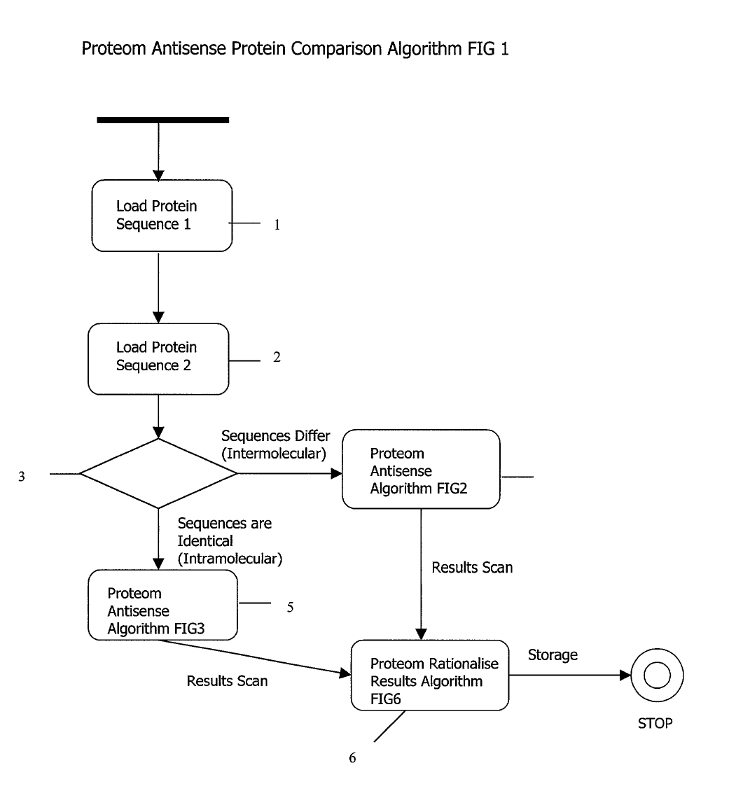

Diagrams describing the algorithms involved in this software are shown in FIGS. 1-5.

### DETAILED DESCRIPTION

1. Overview

The present process is directed toward a computer-based process, a computer-based system and/or a computer program product for analysing antisense relationships between protein or DNA sequences. The method of the embodiment provides a tool for the analysis of protein or i DNA sequences for antisense relationships. This embodiment covers analysis of DNA or protein sequences for intramolecular (within the same sequence) antisense relationships or inter-molecular (between 2 different sequences) antisense relationships. This principle applies whether the sequence contains amino acid information (protein) or DNA information, since the former may be derived from the latter.

The overall process is to facilitate the batch analysis of an entire genome (collection of genes/and or protein sequences) for every possible antisense relationship of both inter- and intra-molecular nature. For the purpose of example it will be described here how a protein sequence database may be analysed by the methods described.

The program runs in two modes. The first mode (Intermolecular) is to select the first protein sequence in the databases and then analyse the antisense relationships between this sequence and all other protein sequences, one at a time. The program then selects the second sequence and repeats this process. This continues until all of the possible relationships have been analysed. The second mode (Intramolecular) is where each protein sequence is analysed for antisense relationships within the same protein and thus each sequence is loaded from the database and analysed in turn for these properties. Both operational modes use the same core algorithms for their processes. The core algorithms are described in detail below.

An example of the output from this process is a list of proteins in the database that contain highly improbable numbers of intramolecular antisense frames of size 10 (frame size is a section of the main sequence, it is described in more detail below).

2. Method

For the purpose of example protein sequence 1 is ATRGRDSRDERSDERTD and protein sequence 2 is GTFRTSREDSTYSGDTDFDE (universal 1 letter amino acid codes used).

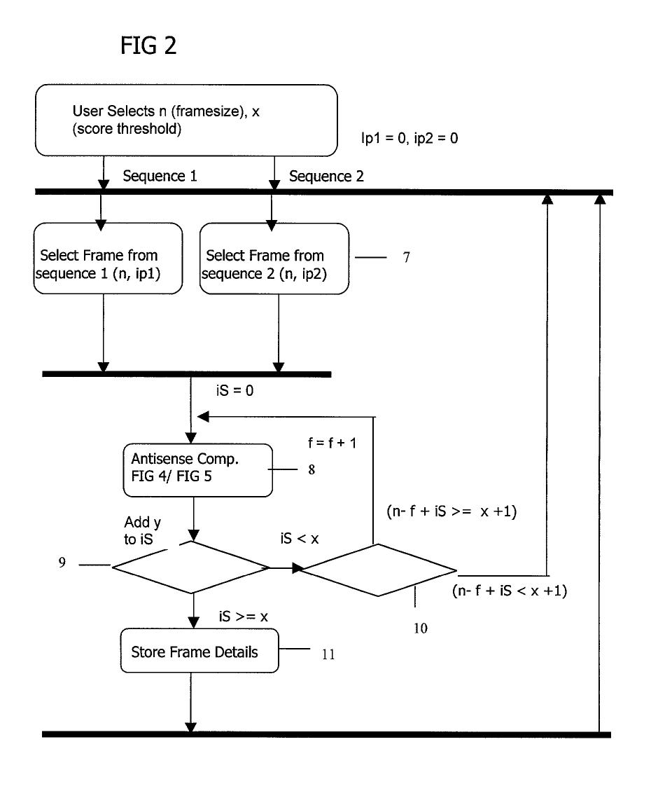

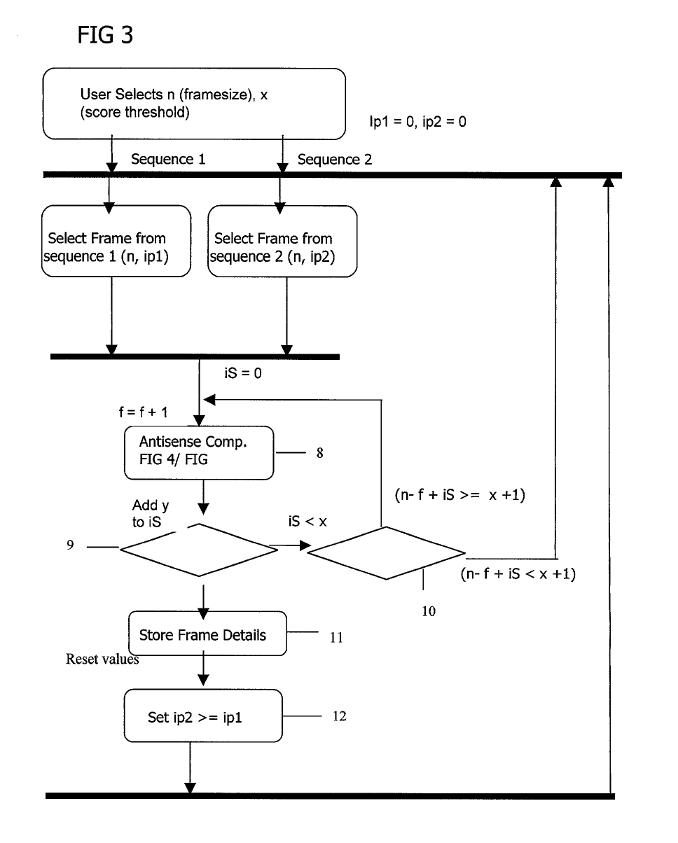

In step 1 (see FIG. 1), a protein sequence, sequence 1, is loaded. The protein sequence consists of an array of universally recognised amino acid one letter codes, e.g. ‘ADTRGSRD’. The source of this sequence can be a database, or any other file type. Step 2, is the same operation as for step 1, except sequence 2 is loaded. Decision step 3 involves comparing the two sequences and determining whether they are identical, or whether they differ. If they differ, processing continues to step 4, described in FIG. 2, otherwise processing continues to step 5, described in FIG. 3.

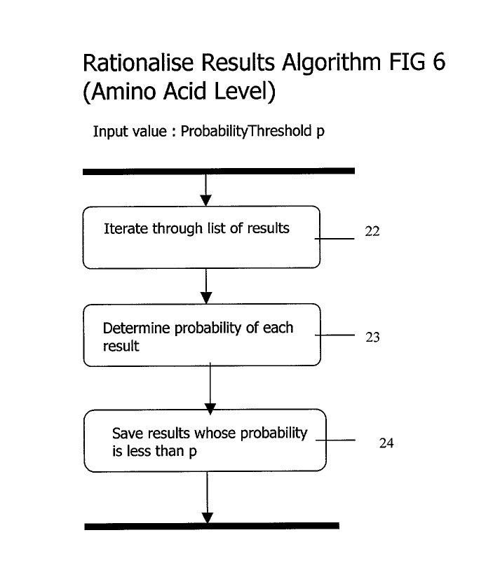

Step 6 analyses the data resulting from either step 4, or step 5, and involves an algorithm described in FIG. 6.

Description of parameters used in FIG. 2

| Name | Description |
|---|---|
| N | Framesize—the number of amino acids that make up each ‘frame’ |
| X | Score threshold—the number of amino acids that have to fulfil the |
| | antisense criteria within a given frame for that frame to be stored |
| | for analysis |
| Y | Score of individual antisense comparison (either 1 or 0) |
| IS | Running score for frame—(sum of y for frame) |
| ip1 | Position marker for Sequence 1—used to track location of selected |
| | frame for sequence 1 |
| ip2 | Position marker for Sequence 2—used to track location of selected |
| | frame for sequence 1 |
| F | Current position in frame |

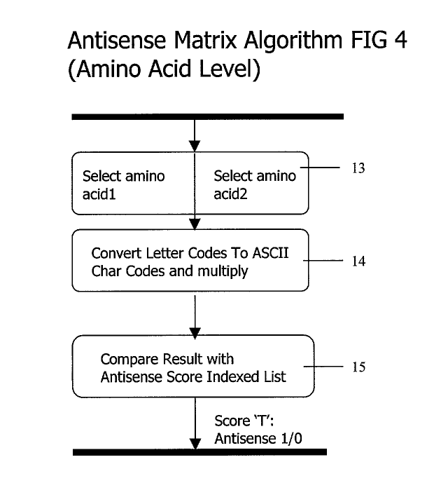

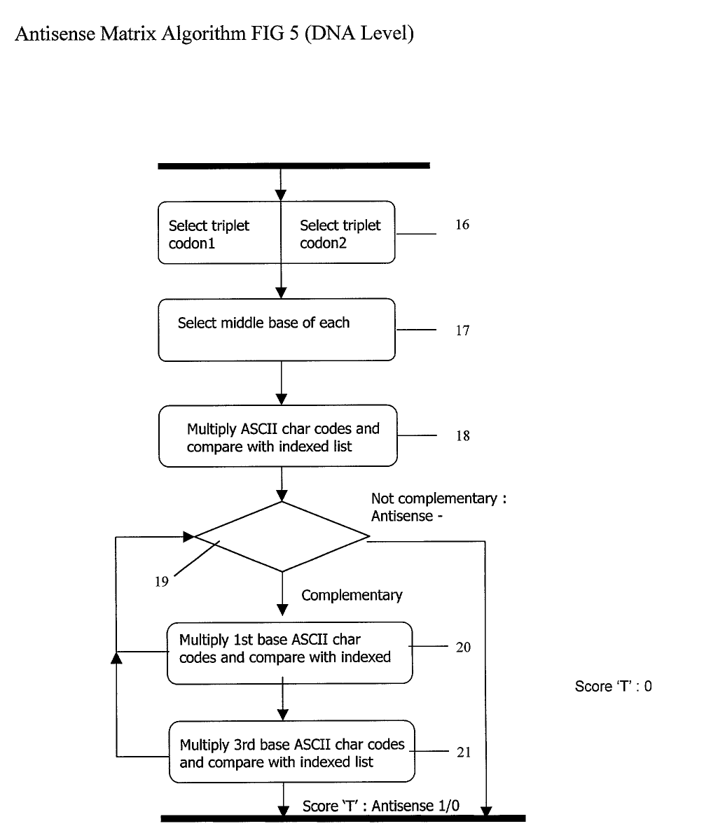

In Step 7, a ‘frame’ is selected for each of the proteins selected in steps 1 and 2. A ‘frame’ is a specific section of a protein sequence. For example, for sequence 1, the first frame of length ‘5’ would correspond to the characters ‘ATRGR’. The user of the program decides the frame length as. an input value. This value corresponds to parameter ‘n’ in FIG. 2. A frame is selected from each of the protein sequences (sequence 1 and sequence 2). Each pair of frames that are selected are aligned and frame position parameter f is set to zero. The first pair of amino acids are ‘compared’ using the algorithm shown in FIG. 4/FIG. 5. The score output from this algorithm (y, either one or zero) is added to a aggregate score for the frame iS. In decision step 9 it is determined whether the aggregate score iS is greater than the Score threshold value (x). If it is then the frame is stored for further analysis. If it is not then decision step 10 is implemented. In decision step 10, it is determined whether it is possible for the frame to yield the score threshold (x). If it can, the frame processing continues and f is incremented such that the next pair of amino acids are compared. If it cannot, the loop exits and the next frame is selected. The position that the frame is selected from the protein sequences is determined by the parameter ip1 for sequence 1 and ip2 for sequence 2 (refer to FIG. 2). Each time steps 7 to 10 or 7 to 11 are completed, the value of ip1 is zeroed and then incremented until all frames of sequence 1 have been analysed against the chosen frame of sequence 2. When this is done, ip2 is then incremented and the value of ip1 is incremented until all frames of sequence 1 have been analysed against the chosen frame of sequence 2. This process repeats and terminates when ip2 is equal to the length of sequence 2. Once this process is complete, sequence 1 is reversed programmatically and the same analysis as described above is repeated. The overall effect of repeating steps 7 to 11 using each possible frame from both sequences is to facilitate step 8, the antisense scoring matrix for each possible combination of linear sequences at a given frame length.

FIG. 3 shows a block diagram of the algorithmic process that is carried out in the conditions described in FIG. 1. Step 12 is the only difference between the algorithms FIG. 2 and FIG. 3. In step 12, the value of ip2 (the position of the frame in sequence 2) is set to at least the value of ip1 at all times since as sequence 1 and sequence 2 are identical, if ip2 is less than ip1 then the same sequences are being searched twice.

FIGS. 4 and 5 describe the process in which a pair of amino acids (FIG. 4) or a pair of triplet codons are assessed for an antisense relationship. The antisense relationships are listed in EXAMPLES 8 and 9. In step 13, the currently selected amino acid from the current frame of sequence 1 and the currently selected amino acid from the current frame of sequence 2 (determined by parameter ‘f’ in FIGS. 2/3) are selected. For example, the first amino acid from the first frame of sequence 1 would be ‘A’ and the first amino acid from the first frame of sequence 2 i would be ‘G’. In step 14, the ASCII character codes for the selected single uppercase characters are determined and multiplied and, in step 15, the product compared with a list of precalculated scores, which represent the antisense relationships in EXAMPLES 8 and 9. If the amino acids are deemed to fulfil the criteria for an antisense relationship (the product matches a value in the precalculated list) then an output parameter ‘T’ is set to 1, otherwise the output parameter is set to zero.

Steps 16-21 relate to the case where the input sequences are DNA/RNA code rather the protein sequence. For example sequence 1 could be AAATTTAGCATG and sequence 2 could be TTTAAAGCATGC. The domain of the current invention includes both of these types of information as input values, since the protein sequence can be decoded from the DNA sequence, in accordance with the genetic code. Steps 16-21 determine antisense relationships for a given triplet codon. In step 16, the currently selected triplet codon for both sequences is ‘read’. For example, for sequence I the first triplet codon of the first frame would be ‘AAA’, and for sequence 2 this would be ‘TTT’. In step 17, the second character of each of these strings is selected. in step 18, the ASCII codes are multiplied and compared, in decision step 19, to a list to find out if the bases selected are ‘complementary’, in accordance with the rules of the genetic code. If they are, the first bases are compared in step 20, and subsequently the third bases are compared in step 21. Step 18 then determines whether the bases are ‘complementary’ or not. If the comparison yields a ‘non-complementary’ value at any step the routine terminates and the output score ‘T’ is set to zero. Otherwise the triplet codons are complementary and the output score T=1.

FIG. 6 illustrates the process of rationalising the results after the comparison of 2 protein or 2 DNA sequences. In step 22, the first ‘result’ is selected. A result consists of information on a pair of frames that were deemed ‘antisense’ in FIG. 2 or 3. This information includes location, length, score (i.e. the sum of scores for a frame) and frame type (forward or reverse, depending on orientation of sequences with respect to one another). In step 23, the frame size, the score values and the length of the parent sequence are then used to calculate the probability of that frame existing. The statistics, which govern the probability of any frame existing, are described in the next section and refer to equations 1-4. If the probability is less than a user chosen value ‘p’, then the frame details are ‘stored’ for inclusion in the final result set (step 24).

### Statistical Basis of Program Operation

The number of complementary frames in a protein sequence can be predicted from appropriate use of statistical theory.

The probability of any one residue fitting the criteria for a complementary relationship with any other is defined by the groupings illustrated in EXAMPLE 8. Thus, depending on the residue in question, there are varying probabilities for the selection of a complementary amino acid. This is a result of an uneven distribution of possible partners. For example possible complementary partners for a tryptophan residue include only proline whilst glycine, serine, cysteine and arginine all fulfil the criteria as complementary partners for threonine. The probabilities for these residues aligning with a complementary match are thus 0.05 and 0.2 respectively. The first problem in fitting an accurate equation to describe the expected number of complementary frames within any sequence is integrating these uneven probabilities into the model. One solution is to use an average value of the relative abundance of the different amino acids in natural sequences. This is calculated by equation 1

v=ΣR*N 1

Where v=probability sum, R=fractional abundance of amino acid in e. coli proteins, N=number of complementary partners specified by genetic code.

This value (p) is calculated as 2.98. The average probability (p) of selecting a complementary amino acid is thus 2.98/20=0.149.

For a single ‘frame’ of size (n) the probability (C) of pairing a number of complementary amino acids (r) can be described by the binomial distribution (equation 2)

$$C = \frac{n !}{( n - r ) ! r !} {{p}^{r} ( 1 - p )}^{n - r} \qquad 2$$

With this information we can predict that the expected number (Ex) of complementary frames in a protein to be:

$$Ex = 2 {( S - n )}^{2} \frac{n !}{( n - r ) ! r !} {{p}^{r} ( 1 - p )}^{n - r} \qquad 3$$

Where S=protein length, n=frame size, r=number of complementary residues required for a frame and p=0.149. If r=n, representing that all amino acids in a frame have to fulfil a complementary relationship, the above equation simplifies to:

Ex=2(S−n)²pⁿ 4

For a population of randomly assembled amino acid chains of a predetermined length we would expect the number of frames fulfilling the complementary criteria in the search algorithm to vary in accordance with a normal distribution.

Importantly, it is possible to standardise results such that given a calculated mean (μ) and standard deviation (σ) for a population it is possible to determine the probability of any specific result occurring. Standardisation of the distribution model is facilitated by the following relation:

$$Z = \frac{X - μ}{σ} \qquad 5$$

Where X is an single value (result) in a population.

If we are considering complementary frames with a single protein structure then the above statistical model requires further analysis. In particular, the possibility exists that a region may be complementary to itself, as indicated in the diagram below.

[image]

Reverse turn motifs within proteins. A region of protein may be complementary to itself In this scenario, A-S, L-K and V-D are complementary partners. A six amino acid wide frame would thus be reported (in reverse orientation). A frame of this type is only specified by half of the residues in the frame. Such a frame is called a reverse turn.

In this scenario, once half of the frame length has been selected with complementary partners, there is a finite probability that those partners are the sequential neighbouring amino acids to those already selected. The probability of this occurring in any protein of any sequence is:

Ex=p^{f}^{^{/2}}(S−f)

Where f is the frame size for analysis, and S is the sequence length and p is the average probability of choosing an antisense amino acid.

The software of the embodiment incorporates all of the statistical models reported above such that it may assess whether a frame qualifies as a forward frame, reverse frame, or reverse turn.

### EXAMPLE 1

### Protein and Nucleotide Sequence Databases Amenable for Analysis Using the Process

Major Nucleic acid databases

| Database | Description | Web site address |
|---|---|---|
| Genbank | The Genbank database is a repository for | Http://www.ncbi. |
| NCBI | nucleotide data. | nlm.nih.gov/ |
| National Center for | The NCBI provides facilities to search for | |
| Biotechnology Information | sequences in Genbank by text or by sequence | |
| | similarity and to submit new sequences. | |
| EMBL | The EMBL database is a repository for nucleotide | http://www.ebi.ac |
| | data. | .uk |
| | The EBI provides facilities to search for | |
| | sequences by text or by sequence similarity and to | |
| | submit new sequences. | |
| DbEST | The dbEST database is a repository for Expressed | http://www.ncbi.n |
| | Sequence Tags (EST) data. | lm.nih.gov/dbES |
| | | T/ |
| Unigene | The Unigene database is a repository for clustered | http://www.ncbi.n |
| | EST data. | lm.nih.gov/UniGe |
| | UniGene is an experimental system for | ne/ |
| | automatically partitioning EST sequences into a | |
| | non-redundant set of gene-oriented clusters. Each | |
| | UniGene cluster contains sequences that represent | |
| | a unique gene, as well as related information such | |
| | as the tissue types in which the gene has been | |
| | expressed and map location. | |
| | Unigene is split up in sections, catagorized by | |
| | species origin. The current three sections are | |
| | Human (hsuinigene), Mouse (mmunigene) and | |
| | Rat (rnunigene) EST clusters. | |
| STACK | STACK is a public database of sequences | http://www.sanbi. |
| | expressed in the human genome. | ac.za/Dbases.html |
| | The STACK project aims to make the most | |
| | comprehensive representation of the sequence of | |
| | each of the expressed genes in the human | |
| | genome, by extensive processing of gene | |
| | fragments to make accurate alignments, highlight | |
| | errors and provide a carefully joined set of | |
| | consensus sequences for each gene. A new | |
| | method to extensively process gene fragments to | |
| | make accurate alignment, prevent errors and | |
| | provide a carefully joined set consensus | |
| | sequences for each gene. | |

Major Protein Sequence databases

| Database | Description | URL |
|---|---|---|
| SWISS-PROT | Curated protein sequence database which strives | http://www.expasy.ch/sprot/sprot |
| | to provide a high level of annotations (such as the | -top.html |
| | description of the function of a protein, its | |
| | domains structure, post-translational | |
| | modifications, variants, etc), a minimal level of | |
| | redundancy and high level of integration with | |
| | other databases. | |
| TrEMBL | Supplement of SWISS-PROT that contains all the | http://www.expasy.ch/sprot/sprot |
| | translations of EMBL nucleotide sequence entries | -top.html |
| | not yet integrated in SWISS-PROT. | |
| OWL | Non-redundant composite of 4 publicly available | http://www.biochem.ucl.ac.uk/bs |
| | primary sources: SWISS-PROT, PIR (1-3), | m/dbbrowser/OWL/OWL.html |
| | GenBank (translation) and NRL-3D. SWISS- | |
| | PROT is the highest priority source, all others | |
| | being compared against it to eliminate identical | |
| | and trivially different sequences. The strict | |
| | redundancy criteria render OWL relatively | |
| | “small” and hence efficient in similarity searches. | |
| PIR Protein | A comprehensive, annotated, and non-redundant | http://pir.georgetown.edu/ |
| Information | set of protein sequence databases in which entries | |
| Resource | are classified into family groups and alignments | |
| | of each group are available. | |
| SPTR | Comprehensive protein sequence database that | http://bioinformer.ebi.ac.uk/newsl |
| | combines the high quality of annotation in | etter/archives/4/sptr.html |
| | SWISS-PROT with the completeness of the | |
| | weekly updated translation of protein coding | |
| | sequences from the EMBL nucleotide database. | |
| NRL_3D | The NRL_3D database is produced by PIR from | http://www- |
| | sequence and annotation information extracted | nbrf.georgetown.edu/pirwww/sea |
| | from the Brookhaven Protein Databank (PDB) of | rch/textnrl3d.html |
| | crystallographic 3D structures. | |

### EXAMPLE 2

### Algorithm Determined Sequence In IL-1 Receptor Binding to IL-1β

The programme identified the antisense region LITVLNI in the interleukin 1 type 1 receptor (IL-1R). The biological relevance of this peptide has been demonstrated and these findings are summarised below:

Program picked out antisense region LITVLNI in the IL-1R receptor.

This peptide was shown to inhibit the biological activity of IL-1β in two independent in vitro bioassays.

The effect is dependent on the peptide sequence.

The same effect is also seen in a Serum Amyloid IL-1 assay (i.e. assay independence).

The peptide was shown to bind directly to IL-1 by using biosensing techniques

[image]

### EXAMPLE 3

### Demonstration of the Utility of the Process when Applied to the Human Genome

1. DNA-Binding Proteins

Sequence-specific DNA binding by proteins controls transcription (Pabo and Sauer, 1992), recombination (Craig, 1988), restriction (Pingoud and Jeltsch, 1997) and replication (Margulies and Kaguni, 1996). Sequence requirements are usually determined by assays that measure the effects of mutations on binding of DNA and amino acid residues implicated in these interactions.

The central role of DNA binding proteins in the cell cycle means they have a key role in cell proliferation, tumour formation and progression.

The identification of anti-sense peptides targetted to such proteins have the potential to be useful targets for the development of therapeutic compounds for the treatment of cancer.

For instance, Koivunen et al., 1999, identified a novel cyclic decapeptide that not only targetted angiogenic (developing) blood vessels but also inhibited the matrix metalloproteinases MMP-2 and MMP-9 (MMP activity is a requirement of tumour growth, angiogenesis and metastasis). The specificity of this novel peptide for MMP-2 and MMP-9 but not other metalloproteinases suggested it might prove useful in tumour therapy. When injected into mice the peptide impeded both growth and invasion of established tumours.

This research demonstrates the potential for using specific peptides as agents for targetting tumours and as anticancer therapies.

2. The Human Major Histocompatibility Complex

The human major histocompatibility complex is associated with more diseases than any other region of the human genome, including most autoimmune conditions (e.g. diabetes and rheumatoid arthritis). A search of OMIM retrieved 187 entries under Major Histocompatibility Complex, associated with phenotypes such as multiple sclerosis, coeliac disease, Graves disease and alopecia.

The first complete sequence of the human MHC region on chromosome 6 has recently been determined (The MHC sequencing consortium, 1999). Over 200 gene loci were identified making this the most gene-dense region of the human genome sequenced so far. Of these, many are of unknown function but at least 40% of the 128 genes predicted to be expressed are involved in immune system function. It also encodes the most polymorphic proteins, the class I and class II molecules, some of which have over 200 allelic variants. This extreme polymorphism is thought to be driven and maintained by the conflict between the immune system and infectious pathogens.

The importance of this region to human disease makes it an ideal target for analysis to identify novel therapeutic peptides.

### EXAMPLE 4

The human genome, which is estimated to contain between 80,000 and 140,000 genes was screened for intermolecular peptides using the method described in patent application number GB 9927485.4, filed Nov. 19, 1999. The gene, database accession number, its predicted interacting peptides and their position within the coding sequence of the gene are shown in the attached sequence listing: SEQ ID Nos. [1-3622].

### EXAMPLE 5

### Derivation of Daughter Sequences from Parent Sequences

For each pair of ‘frames’ of amino acids which are deemed a ‘hit’ by the algorithm the current invention includes derived pairs of composite daughter sequences of shorter frame lengths which automatically fulfil the same ‘complementary’ relationship.

For example, there is a complementary frame of size 10 between genes (inter-molecular) CBFA2 and ACTR3 of Homo Sapien.:

| GENE1 | GENE2 | Sequence 1 | Location | Sequence 2 | Location | Score |
|---|---|---|---|---|---|---|
| CBFA2 | ACTR3 | DLRFVGRSGR | 133-142 | PTAAPDKTEV | 77-86 | 10 |

One embodiment of the invention covers the derivation of the following sequences at frame length of 5:

| | | Seq- | | Seq- | Loc- | |
|---|---|---|---|---|---|---|
| GENE | GENE2 | uence 1 | Location | uence 2 | ation | Score |
| CBFA2 | ACTR3 | DLRFV | 133-137 | VETKD | 77-81 | 5 |
| CBFA2 | ACTR3 | LRFVG | 134-138 | ETKDP | 78-82 | 5 |
| CBFA2 | ACTR3 | RFVGR | 135-139 | TKDPA | 79-83 | 5 |
| CBFA2 | ACTR3 | FVGRS | 136-140 | KDPAA | 80-84 | 5 |
| CBFA2 | ACTR3 | VGRSG | 137-141 | DPAAT | 81-85 | 5 |
| CBFA2 | ACTR3 | GRSGR | 138-142 | PAATP | 82-86 | 5 |

One embodiment of the invention covers the derivation of the following sequences at frame length of 6:

| GENE | GENE2 | Sequence 1 | Location | Sequence 2 | Location | Score |
|---|---|---|---|---|---|---|
| CBFA2 | ACTR3 | DLRFVG | 133-138 | VETKDP | 77-82 | 6 |
| CBFA2 | ACTR3 | LRFVGR | 134-139 | ETKDPA | 78-83 | 6 |
| CBFA2 | ACTR3 | RFVGRS | 135-140 | TKDPAA | 79-84 | 6 |
| CBFA2 | ACTR3 | FVGRSG | 136-141 | KDPAAT | 80-85 | 6 |
| CBFA2 | ACTR3 | VGRSGR | 137-142 | DPAATP | 81-86 | 6 |

One embodiment of the invention covers the derivation of the following sequences at frame length of 7:

| GENE | GENE2 | Sequence 1 | Location | Sequence 2 | Location | Score |
|---|---|---|---|---|---|---|
| CBFA2 | ACTR3 | DLRFVGR | 133-139 | VETKDPA | 77-83 | 7 |
| CBFA2 | ACTR3 | LRFVGRS | 134-140 | ETKDPAA | 78-84 | 7 |
| CBFA2 | ACTR3 | RFVGRSG | 135-141 | TKDPAAT | 79-85 | 7 |
| CBFA2 | ACTR3 | FVGRSGR | 136-142 | KDPAATP | 80-86 | 7 |

One embodiment of the invention covers the derivation of the following sequences at frame length of 8:

| GENE | GENE2 | Sequence 1 | Location | Sequence 2 | Location | Score |
|---|---|---|---|---|---|---|
| CBFA2 | ACTR3 | DLRFVGRS | 133-140 | VETKDPAA | 77-84 | 8 |
| CBFA2 | ACTR3 | LRFVGRSG | 134-141 | ETKDPAAT | 78-85 | 8 |
| CBFA2 | ACTR3 | RFVGRSGR | 135-142 | TKDPAATP | 79-86 | 8 |

One embodiment of the invention covers the derivation of the following sequences at frame length of 9:

| GENE | GENE2 | Sequence 1 | Location | Sequence 2 | Location | Score |
|---|---|---|---|---|---|---|
| CBFA2 | ACTR3 | DLRFVGRSG | 133-141 | VETKDPAAT | 77-85 | 9 |
| CBFA2 | ACTR3 | LRFVGRSGR | 134-142 | ETKDPAATP | 78-86 | 9 |

### EXAMPLE 6

The human genome, which is estimated to contain between 80,000 and 140,000 genes was screened for intramolecular peptides using the method described in patent application number GB 9927485.4, filed Nov. 19, 1999. The gene, database accession number, its predicted interacting peptides and their position within the coding sequence of the gene are shown in the attached sequence listing: SEQ ID Nos. [3624-4203].

### EXAMPLE 7

### Derivation of Daughter Sequences from Parent Sequences

For each pair of ‘frames’ of amino acids which are deemed a ‘hit’ by the algorithm the current invention includes derived pairs of composite daughter sequences of shorter frame lengths which automatically fulfil the same ‘complementary’ relationship.

For example, gene ADRAIB in Homo Sapiens contains the following intra-molecular complementary relationship of frame length 10:

| GENE | Sequence 1 | Location | Sequence 2 | Location | Score |
|---|---|---|---|---|---|
| ADRA1B | GGGSAGGAAP | 28-37 | GGGSAGGAAP | 28-37 | 10 |

One embodiment of the invention covers the derivation of the following sequences at frame length of 5:

| GENE | Sequence 1 | Location | Sequence 2 | Location | Score |
|---|---|---|---|---|---|
| ADRA1B | GGGSA | 28-32 | PAAGG | 37-33 | 5 |
| ADRA1B | GGSAG | 29-33 | AAGGA | 36-32 | 5 |
| ADRA1B | GSAGG | 30-34 | AGGAS | 35-31 | 5 |
| ADRA1B | SAGGA | 31-35 | GGASG | 34-30 | 5 |
| ADRA1B | AGGAA | 32-36 | GASGG | 33-29 | 5 |
| ADRA1B | GGAAP | 33-37 | ASGGG | 32-28 | 5 |

One embodiment of the invention covers the derivation of the following sequences at frame length of 6:

| GENE | Sequence 1 | Location | Sequence 2 | Location | Score |
|---|---|---|---|---|---|
| ADRA1B | GGGSAG | 28-33 | PAAGGA | 37-32 | 6 |
| ADRA1B | GGSAGG | 29-34 | AAGGAS | 36-31 | 6 |
| ADRA1B | GSAGGA | 30-35 | AGGASG | 35-30 | 6 |
| ADRA1B | SAGGAA | 31-36 | GGASGG | 34-29 | 6 |
| ADRA1B | AGGAAP | 32-37 | GASGGG | 33-28 | 6 |

One embodiment of the invention covers the derivation of the following sequences at frame length of 7:

| GENE | Sequence 1 | Location | Sequence 2 | Location | Score |
|---|---|---|---|---|---|
| ADRA1B | GGGSAGG | 28-34 | PAAGGAS | 37-31 | 7 |
| ADRA1B | GGSAGGA | 29-35 | AAGGASG | 36-30 | 7 |
| ADRA1B | GSAGGAA | 30-36 | AGGASGG | 35-29 | 7 |
| ADRA1B | SAGGAAP | 31-37 | GGASGGG | 34-28 | 7 |

One embodiment of the invention covers the derivation of the following sequences at frame length of 8:

| | | Loc- | | | |
|---|---|---|---|---|---|
| GENE | Sequence 1 | ation | Sequence 2 | Location | Score |
| ADRA1B | GGGSAGGA | 28-35 | PAAGGASG | 37-30 | 8 |
| ADRA1B | GGSAGGAA | 29-36 | AAGGASGG | 36-29 | 8 |
| ADRA1B | GSAGGAAP | 30-37 | AGGASGGG | 35-28 | 8 |

One embodiment of the invention covers the derivation of the following sequences at frame length of 9:

| GENE | Sequence 1 | Location | Sequence 2 | Location | Score |
|---|---|---|---|---|---|
| ADRA1B | GGGSAGGAA | 28-36 | PAAGGASGG | 37-29 | 9 |
| ADRA1B | GGSAGGAAP | 29-37 | AAGGASGGG | 36-28 | 9 |

### EXAMPLE 8

### The Amino Acid Pairings Resulting from Reading the Anticodon for Naturally Occuring Amino Acid Residues in the 5′-3′ Direction

| | | | | | | | Comple- |
|---|---|---|---|---|---|---|---|
| | | Comple- | | | | Comple- | mentary |
| Amino | co- | mentary | Complementary | Amino | co- | mentary | Amino |
| Acid | don | codon | Amino acid | Acid | don | codon | acid |
| Alanine | GCA | UGC | Cysteine | Serine | UCA | UGA | Stop |
| | GCG | CGC | Arginine | | UCC | GGA | Glycine |
| | GCC | GGC | Glycine | | UCG | CGA | Arginine |
| | GCU | AGC | Serine | | UCU | AGA | Arginine |
| | | | | | AGC | GCU | Alanine |
| | | | | | AGU | ACU | Threonine |
| Arginine | CGG | CCG | Proline | Glutamine | CAA | UUG | Leucine |
| | CGA | UCG | Serine | | CAG | CUG | Leucine |
| | CGC | GCG | Alanine | | | | |
| | CGU | ACG | Threonine | | | | |
| | AGG | CCU | Proline | | | | |
| | AGA | UCU | Serine | | | | |
| Aspartic Acid | GAC | GUC | Valine | Glycine | GGA | UCC | Serine |
| | GAU | AUC | Isoleucine | | GGC | GCC | Alanine |
| | | | | | GGU | ACC | Threonine |
| | | | | | GGG | CCC | Proline |
| Asparagine | AAC | GUU | Valine | Histidine | CAC | GUG | Valine |
| | AAU | AUU | Isoleucine | | CAU | AUG | Methionine |
| Cysteine | UGU | ACA | Threonine | Isoleucine | AUA | UAU | Tyrosine |
| | UGC | GCA | Alanine | | AUC | GAU | Aspartic |
| | | | | | AUU | AAU | acid |
| | | | | | | | Asparagine |
| Glutamic | GAA | UUC | Phenylalanine | Leucine | CUG | CAG | Glutamine |
| Acid | GAG | CUC | Leucine | | CUC | GAG | Glutamic |
| | | | | | CUU | AAG | acid |
| | | | | | UUA | UAA | Lysine |
| | | | | | CUA | UAG | Stop |
| | | | | | UUG | CAA | Stop |
| | | | | | CUG | CAG | Glutamine |
| | | | | | | | Glutamine |
| Lysine | AAA | UUU | Phenylalanine | Threonine | ACA | UGU | Cysteine |
| | AAG | CUU | Leucine | | ACG | CGU | Arginine |
| | | | | | ACC | GGU | Glycine |
| | | | | | ACU | AGU | Serine |
| Methionine | AUG | CAU | Histidine | Tryptophan | UGG | CCA | Proline |
| Phenylalanine | UUU | AAA | Lysine | Tyrosine | UAC | GUA | Valine |
| | UUC | GAA | Glutamic Acid | | UAU | AUA | Isoleucine |
| Proline | CCA | UGG | Tryptophan | Valine | GUA | UAC | Tyrosine |
| | CCC | GGG | Glycine | | GUG | CAC | Histidine |
| | CCU | AGG | Arginine | | GUC | GAC | Aspartic |
| | CCG | CGG | Arginine | | GUU | AAC | Acid |
| | | | | | | | Asparagine |

### EXAMPLE 9

The relationships between amino acids and the residues encoded in the complementary strand reading 3′-5′

| | | | | | | | Comple- |
|---|---|---|---|---|---|---|---|
| | | Comple- | | | | Comple- | mentary |
| Amino | co- | mentary | Complementary | Amino | co- | mentary | Amino |
| Acid | don | codon | Amino acid | Acid | don | codon | acid |
| Alanine | GCA | CGU | Arginine | Serine | UCA | AGU | Serine |
| | GCG | CGC | | | UCC | AGG | Arginine |
| | GCC | CGG | | | UCG | AGC | Serine |
| | GCU | CGA | | | UCU | AGA | Arginine |
| | | | | | AGC | UCG | Serine |
| | | | | | AGU | UCA | Serine |
| Arginine | CGG | GCC | Alanine | Glutamine | CAA | GUU | Valine |
| | CGA | GCU | Alanine | | CAG | GUC | Valine |
| | CGC | GCG | Alanine | | | | |
| | CGU | GCA | Alanine | | | | |
| | AGG | UCC | Serine | | | | |
| | AGA | UCU | Serine | | | | |
| Aspartic Acid | GAC | GUC | Valine | Glycine | GGA | CCU | Proline |
| | GAU | AUC | Isoleucine | | GGC | CCG | Proline |
| | | | | | GGU | CCA | Proline |
| | | | | | GGG | CCC | Proline |
| Asparagine | AAC | UUG | Leucine | Histidine | CAC | GUG | Valine |
| | AAU | UUA | Leucine | | CAU | GUA | Valine |
| Cysteine | UGU | ACA | Threonine | Isoleucine | AUA | UAU | Tyrosine |
| | UGC | ACG | Threonine | | AUC | UAG | Stop |
| | | | | | AUU | UAA | Stop |
| Glutamic | GAA | CUU | Leucine | Leucine | CUG | GAC | Asp |
| Acid | GAG | CUG | Leucine | | CUC | GAG | Glutamic |
| | | | | | CUU | GAA | acid |
| | | | | | UUA | AAU | Glutamic |
| | | | | | CUA | GAU | Acid |
| | | | | | UUG | AAC | Asparagine |
| | | | | | CUG | GAC | Aspartic |
| | | | | | | | Acid |
| | | | | | | | Asparagine |
| | | | | | | | Aspartic |
| | | | | | | | Acid |
| Lysine | AAA | UUU | Phenylalanine | Threonine | ACA | UGU | Cysteine |
| | AAG | UUC | Phenylalanine | | ACG | UGC | Cysteine |
| | | | | | ACC | UGG | Tryptophan |
| | | | | | ACU | UGA | Stop |
| Methionine | AUG | UAC | Tyrosine | Tryptophan | UGG | ACC | Threonine |
| Phenylalanine | UUU | AAA | Lysine | Tyrosine | UAC | AUG | Methionine |
| | UUC | AAG | Lysine | | UAU | AUA | Isoleucine |
| Proline | CCA | GGU | Glycine | Valine | GUA | CAU | Histidine |
| | CCC | GGG | Glycine | | GUG | CAC | Histidine |
| | CCU | GGA | Glycine | | GUC | CAG | Glutamine |
| | CCG | GGC | Glycine | | GUU | CAA | Glutamine |

### REFERENCES

All publications, patents, and patent applications cited are hereby incorporated by reference in their entirety.

Baranyi L, Campbell W, Ohshima K, Fujimoto S, Boros M and Okada H. 1995. The antisense homology box: a new motif within proteins that encodes biologically active peptides. Nature Medicine. 1:894-901.

Craig, N. L. 1998. The mechanism of conservative site-specific recombination. Annu. Rev. Genet. 22: 77-105.

Gaasterland T. 1998. Structural genomics: Bioinformatics in the driver's seat. Nature Biotechnology 16: 645-627.

Goldstein D J. 1998. An unacknowledged problem for structural genomics? Nature Biotechnology 16: 696-697.

Koivunen E, Arap W, Valtanen H, Rainisalo A, Medina O P, Heikkila P, Kantor C, Gahmberg C G, Salo T, Konttinen Y T, Sorsa T, Ruoslahti E, Pasqualini R. 1999. Tumor targeting with a selective gelatinase inhibitor. Nat Biotechnol. 17: 768-74.

Margulies, C. & Kaguni, J. M. 1996. Ordered and sequential binding of DNA protein to oriC, the chromosomal origin of Escherichia coli. J. Biol. Chem. 271: 17035-17040.

The MHC sequencing consortium. 1999. Complete sequence and gene map of a human major histocompatibility complex. Nature 401:921-3.

Pabo, C. O. & Sauer, R. T. 1992. Transcription factors: structural families and principles of DNA recognition. Annu. Rev. Biochem. 61: 1053-1095.

Pingoud, A. & Jeltsch, A. 1997. Recognition and cleavage of DNA by type-II restriction endonucleases. Eur. J. Biochem. 246: 1-22.

Sansom C. 1998. Extending the boundaries of molecular modelling. Nature Biotechnology 16: 917-918.

Stryer L. Biochmistry. 4^{th} Edition. Freeman and Company, New York 1997.

We claim:

1. A set of peptide ligands; said set consisting of specific complementary peptides to proteins encoded by genes of the human genome.

2. A set of peptide ligands according to claim 1, wherein the sequences of the peptides in the set are intra-molecular complementary peptide sequences.

3. A set of peptide ligands according to claim 1, wherein the sequences of the peptides in the set are inter-molecular complementary peptide sequences.

4. A novel peptide having a sequence which is a member of a set according to any preceding claim, capable of antagonising or agonising a specific interaction of a protein with another protein or receptor.

5. Use of a set of peptides according to any of claims 1 to 3 in an assay for screening and identification of one or more peptides according to claim 4.

6. Use according to claim 5 wherein the identified peptide(s) is a drug candidate.

7. Use according to claim 5 wherein the identified peptide(s) is a pro-drug.

8. A partly or wholly non-peptide mimetic of a peptide drug candidate or pro-drug according to claim 4, 6 or 7, identified by use of the set of peptides according to claim 5.

9. A method for processing sequence data comprising the steps of;
selecting a first protein sequence and a second protein sequence;
selecting a frame size corresponding to a number of sequence elements
such as amino acids or triplet codons, a score threshold, and a frame existence probability threshold;
comparing each frame of the first sequence with each frame of the second sequence by comparing pairs of sequence elements at corresponding positions within each such pair of frames to evaluate a complementary relationship score for each pair of frames;
storing details of any pairs of frames for which the score equals or exceeds the score threshold;
evaluating for each stored pair of frames the probability of the existence of that complementary pair of frames existing, on the basis of the number of possible complementary sequence elements existing for each sequence element in the pair of frames; and
discarding any stored pairs of frames for which the evaluated probability is greater than the probability threshold; wherein each frame is a peptide sequence of defined length.

10. A method according to claim 9, in which the first sequence is identical to the second sequence and a frame at a given position in the first sequence is only compared with frames in the second sequence at the same given position or at later positions in the second sequence, in order to eliminate repetition of comparisons.

11. A method according to claim 9 or 10, in which the sequence elements at corresponding positions within each of a pair of frames are compared sequentially, each such pair of sequence elements generating a score which is added to an aggregate score for the pair of frames.

12. A method according to claim 11, in which if the aggregate score reaches the score threshold before all the pairs of sequence elements in the pair of frames have been compared, details of the pair of frames are immediately stored and a new pair of frames is selected for comparison.

13. A method according to any preceding claim, in which the sequence elements are amino acids and pairs of amino acids are compared by using an antisense score list.

14. A method according to any of claims 9 to 12, in which the sequence elements are triplet codons and pairs of codons in corresponding positions within each of the pairs of triplet codons are compared by using an antisense score list.

15. A method for processing sequence data substantially as described herein with reference to FIGS. 1 to 6.

16. A pair of frames or a list of pairs of frames being the product of the method of any of claims 9 to 15, optionally carried on a computer-readable medium.

17. A frame being the product of the method of any of claims 9 to 15, optionally carried on a computer-readable medium.

18. A peptide, pair of complementary peptides, or set of peptides, being the peptide(s) having the sequence of the frame(s) of claims 16 or 17.

19. A method for identifying a peptide drug candidate or pro-drug, which method includes the steps of (i) identifying a set of specific complementary peptides according to any of claims 1 to 4; (ii) screening the set for specific protein interaction activity; and (iii) identifying one or more peptide(s) according to claim 5.
